(19) United States
(12) Patent Application Publication
(10) Pub. No.: US 2020/0038001 A1
(43) Pub. Date: Feb. 6, 2020

(54) TISSUE REMOVAL AND COLLECTION DEVICES

(71) Applicant: Mayo Foundation for Medical Education and Research, Rochester, MN (US)

(72) Inventors: Jesse S. Voss, Rochester, MN (US); Benjamin R. Kipp, Byron, MN (US); Luke Stangler, Waseca, MN (US)

(21) Appl. No.: 16/527,850

(22) Filed: Jul. 31, 2019

Related U.S. Application Data
(60) Provisional application No. 62/713,356, filed on Aug. 1, 2018.

(51) Int. Cl. A61B 10/00; G01N 1/06

(52) U.S. Cl. CPC A61B 10/0096; G01N 1/30; G01N 2001/061; G01N 1/06

(57) ABSTRACT

This document relates to methods and materials for improving tissue removal and collection. For example, this document relates to methods and devices for collection of tissue samples from slides.

### CROSS-REFERENCE TO RELATED APPLICATIONS

This application claims the benefit of U.S. Provisional Application Ser. No. 62/713,356, filed Aug. 1, 2018. The disclosure of the prior application is considered part of (and is incorporated by reference in) the disclosure of this application.

### BACKGROUND

### 1. Technical Field

This document relates to methods and materials for improving tissue removal and collection. For example, this document relates to methods and devices for collection of tissue samples from slides.

### 2. Background Information

Molecular genetic testing of solid tumor samples requires the manual dissection of paraffin-embedded tumor tissue from one or more slides (e.g., 1-10). Sometimes, these slides are unstained. To collect the samples, a blade, such as a scalpel, is used to create small flecks of tissue. The process of collecting the small flecks of tissue can be both time consuming and laborious. Further, scalpels can be a sharps hazard requiring safety precautions and disposals. In addition, this method can lead to contamination of the sample when placing the tissue sample in a microcentrifuge tube for nucleic acid extraction. This can also lead to sample loss, which may require additional extraction. Additional extraction may cause a patient to need to undergo additional surgical procedures to obtain more tissue for molecular testing. The collection of tissue samples can take approximately 10 minutes per case, with more difficult cases taking 20 minutes or more.

Some labs perform over 20,000 assays that require manual dissection of tissue from slides annually. This manual dissection can require a substantial amount of time. In addition, there is an estimated >500,000 new cancer cases diagnosed annually that require molecular genetics testing.

### SUMMARY

This document describes methods and materials for improving tissue removal and collection. For example, this document describes methods and devices for collection of tissue samples from slides.

In one aspect, this disclosure is directed to a sample collection device. The sample collection device includes an elongated shaft defining an internal lumen. The elongated shaft includes a proximal end and a distal end, where the proximal end of the elongated shaft is configured to couple with a suction source. The sample collection device also includes a cap defining an internal lumen. The cap includes a proximal end and a distal end, where the internal lumen of the cap is fluidly coupled to the internal lumen of the elongated shaft, where the proximal end of the cap is configured to couple to the distal end of the elongated shaft, and where the distal end of the cap includes a blade. The sample collection device includes a filter column configured to be secured inside the internal lumen of the elongated shaft via the cap.

In some cases, the second end of the elongated shaft and the first end of the cap can be threaded. In some cases, the filter column can include a filter member. In some cases, the filter member can be located on a distal end of the filter column. In some cases, the filter column can be configured to collect a tissue sample. In some cases, the proximal end of the cap can be configured to removably couple to the filter column. In some cases, the proximal end of the filter column can be configured to removably couple to the proximal end of the cap. In some cases, the sample can be a tissue sample. In some cases, the filter assembly can be unidirectionally configured to couple to the elongated shaft. In some cases, the filter can be compressed between male and female housing components. In some cases, the filter assembly can allow for bidirectional flow. In some cases, the cap can provide dissection capabilities through a flat blade. In some cases, the cap can provide dissection capabilities through a circumferential blade. In some cases, the cap can provide dissection capabilities through a point. In some cases, the second end of the elongated shaft and the first end of the cap can be compression fitted. In some cases, the first end of the elongated shaft and the vacuum attachment can be compression fitted. In some cases, the filter pores can retain targeted assay components.

In another aspect, this disclosure is directed to a method of collecting a sample. The method includes inserting a filter column into a distal end of internal lumen of an elongated shaft and securing a cap comprising a blade onto a distal end of the elongated shaft.

In some cases, the method can include coupling a suction source to a proximal end of the elongated shaft. In some cases, the method can include using the blade to remove portions of the sample. In some cases, the method can include applying suction to the internal lumen to suck the portions of the sample into the filter column. In some cases, portions of the sample can be caught in a filter member located in the filter column. In some cases, the method can include removing the filter column from the internal lumen with portions of the sample inside the filter column.

In yet another aspect, this disclosure is directed to a method of collecting a sample. The method includes scraping the sample using a blade to create portions of the sample, and sucking the portions into a filter column of a tissue collection device. The tissue collection device includes an elongated shaft defining an internal lumen and includes a proximal end and a distal end, where the proximal end of the elongated shaft is configured to couple to a suction source. The tissue collection device also includes a cap defining an internal lumen and includes a proximal end and a distal end, where the proximal end of the cap is configured to couple to the distal end of the elongated shaft, and where the distal end of the cap comprises a blade. The tissue collection device also includes a filter column configured to be secured inside the internal lumen of the elongated shaft via the cap.

In some cases, the method can include trapping portions of the sample in the filter column. In some cases, the method can include removing the cap from the elongated shaft. In some cases, the method can include removing the filter column from the internal lumen of the elongated shaft. In some cases, the method can include inserting the filter column into a centrifuge tube. In some cases, the method can include performing molecular testing in the filter column. In some cases, the method can include rotating the tissue collection device about a flat blade for varied acuity.

Particular embodiments of the subject matter described in this document can be implemented to realize one or more of the following advantages. The methods and devices described herein can reduce the amount of time and effort involved in scraping slides. Time can be saved by collecting the tissue sample flakes into the filter column using a vacuum and then further using the filter column to pass solutions through (e.g., using gravity). Further, the methods and devices can reduce the risk of contamination of tissue samples. In addition, there can be an increase in efficiency of tissue collection such that additional procedures do not need to be conducted to collect more samples.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a perspective view of a user collecting a tissue sample, in accordance with some embodiments provided herein.

FIG. 2 is a side view of a tissue collection device, in accordance with some embodiments provided herein.

FIG. 3 is an exploded view of the tissue collection device of FIG. 2, in accordance with some embodiments provided herein.

FIGS. 4 and 5 are perspective views of a cap for the tissue collection device of FIG. 2, in accordance with some embodiments provided herein.

FIGS. 6 and 7 are perspective views of a second cap for the tissue collection device of FIG. 2, in accordance with some embodiments provided herein.

FIG. 8 is a perspective view of a second tissue collection device, in accordance with some embodiments provided herein.

FIG. 9 is an exploded view of the second tissue collection device of FIG. 8, in accordance with some embodiments provided herein.

FIG. 10 is a side view of an elongated shaft of the tissue collection device of FIG. 9, in accordance with some embodiments provided herein.

FIGS. 11 and 12 are side views of components of a filter assembly of the tissue collection device of FIG. 9, in accordance with some embodiments provided herein.

FIGS. 13-16 are various views of a cap shown in FIG. 8, in accordance with some embodiments provided herein.

FIG. 17 is a perspective view of a third tissue collection device, in accordance with some embodiments provided herein.

FIG. 18 is an exploded view of the tissue collection device of FIG. 17, in accordance with some embodiments provided herein.

FIG. 19 is a side view of an elongated shaft of the tissue collection device of FIG. 18, in accordance with some embodiments provided herein.

FIGS. 20 and 21 are side views of components of a filter assembly of the tissue collection device of FIG. 18, in accordance with some embodiments provided herein.

FIGS. 22-25 are various views of a cap shown in FIG. 17, in accordance with some embodiments provided herein.

Like reference numbers represent corresponding parts throughout.

### DETAILED DESCRIPTION

This document describes methods and materials for improving tissue removal and collection. For example, this document describes methods and devices for collection of tissue samples from slides.

Molecular genetic testing of solid tumor samples requires the manual dissection of paraffin embedded tumor tissue from a plurality of slides (e.g., 1-10). The process of collecting the small flecks of tissue can be both time consuming and laborious. In addition, this method can lead to contamination of the sample when placing the tissue sample in a microcentrifuge tube for nucleic acid extraction. The collection of tissue samples can take approximately 10 minutes per case, with more difficult cases taking 20 minutes or more. Some labs perform over 20,000 assays that require manual dissection of tissue from slides annually, this can require a substantial amount of time. The methods and devices described herein can reduce time required to collect tissue samples (e.g., for molecular testing) and reduce the risk of contamination of tissue samples.

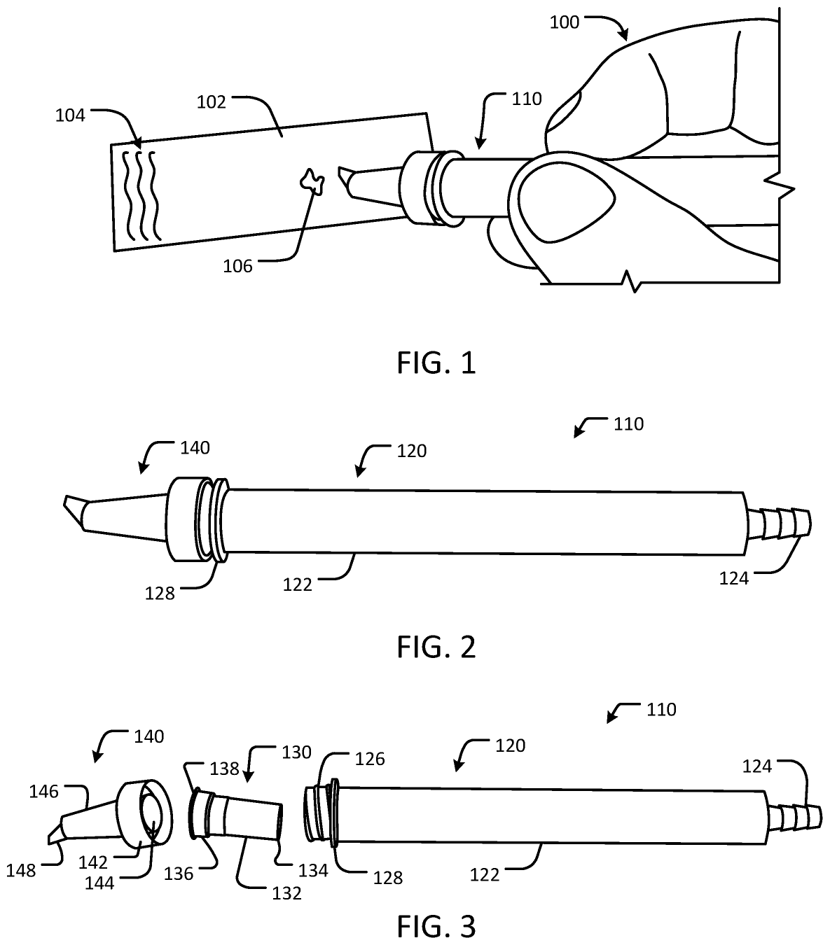

Referring to FIG. 1, a user 100 is removing a tissue sample 106 using a tissue collection device 110. Tissue sample 106 can be removed from a slide 102. In some cases, slide 102 can include a label 104. In some cases, label 104 can include information relating to the patient, sample number, a type of cancer associated with the patient, etc. In some cases, the tissue sample 106 can be unstained. In some cases, the tissue sample 106 is stained. In some cases, a single slide 102 can be stained with hematoxylin and eosin stain (H&E stain) and tumor areas can be circled by a pathologist. In some cases, a technologist can remove the tissue sample 106 from an unstained slide 102 in areas that correspond to the marked tumor areas on the stained slide. The flakes removed from tissue sample 106 can be collected and deposited into a microcentrifuge tube.

In some cases, once the tissue is collected, xylene (e.g., 1 mL) can be added to the tissue flakes in the microcentrifuge to remove paraffin wax. In some cases, the tube can be centrifuged (e.g., for approximately 1 minute) to collect tissue flakes at the bottom of the tube. In some cases, the xylene can be removed with a manual pipette. In some cases, ethanol (e.g., 1 mL) can be added to the tissue sample containing xylene to remove the xylene residue from the tissue sample. In some cases, the tube can be centrifuged (e.g., for approximately 1 minute) to collect tissue flakes at the bottom of the tube. In some cases, the ethanol can be removed with a manual pipette. In some cases, a protein enzyme solution can be added to the dissolved tissue sample. In some cases, the dissolved tissue solution can be used for DNA and/or RNA extraction.

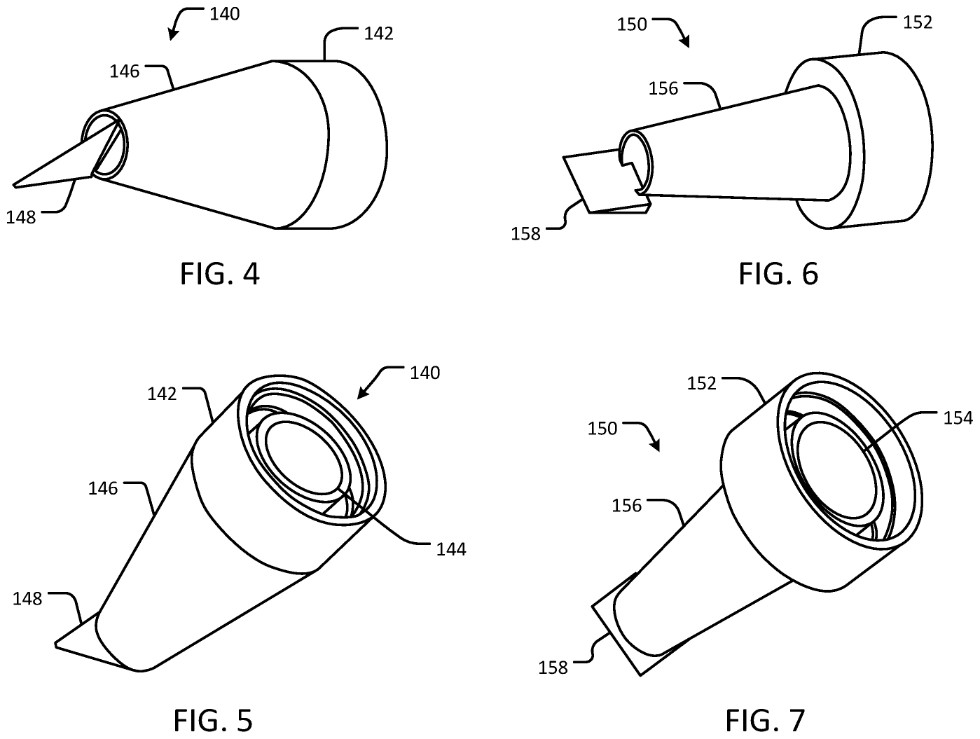

Referring to FIGS. 2 and 3, tissue collection device 110 can include an elongated shaft 120, a filter column 130, and a cap 140. Cap 140 will be described in further detail with respect to FIGS. 4 and 5.

Elongated shaft 120 can include a gripping portion 122, a vacuum coupler 124, a threaded portion 126, and a stop 128. Elongated shaft 120 can be made of polypropylene, xylene-resistant material, ethanol-resistant material, DNase-free material, and/or RNase-free material.

Gripping portion 122 can be cylindrical. In some cases, gripping portion 122 can be a rectangular prism, a triangular prism, or other prism form. In some cases, gripping portion 122 can be sized to comfortably fit in a hand of user 100. Gripping portion 122 can define an internal lumen (not shown). In some cases, gripping portion 122 can include at least one of ridges, texture, indentations, and/or a portion of different material. In some cases, these features can increase coupling and/or comfort between the user 100 and the tissue collection device 110. Vacuum coupler 124 can be located at a proximal end of gripping portion 122.

Vacuum coupler 124 can be configured to receive a tube or other component of a vacuum, or other suction device. Vacuum coupler 124 can allow vacuum suction to be provided to the tissue collection device 110 to aid in collection of tissue. In some cases, the tube of a vacuum can be fitted around vacuum coupler 124. Vacuum coupler 124 can have a barbed design. For example, vacuum coupler can be a series of elements with a conical shape, each with a diameter increasing as each element extends distally, as shown in FIGS. 2 and 3. In some cases, vacuum coupler 124 can define an internal lumen. In some cases, the internal lumen of vacuum coupler 124 is in fluid communication with the internal lumen of gripping portion 122. In some cases, vacuum coupler 124 can threaded for a screw-type connection with a vacuum or suction device.

Threaded portion 126 can be located at a distal end of gripping portion 122. In some cases, threaded portion 126 can include threads to allow the gripping portion 122 to couple to cap 140. In some cases, threaded portion 126 can have a diameter substantially similar to that of gripping portion 122. In some cases, threaded portion 126 can have a diameter larger or small than the diameter of gripping portion 122. In some cases, threaded portion 126 can be threaded to accommodate connection to a variety of caps 140, such that different configurations of cap styles can attach to threaded portion 126. In some cases, threaded portion 126 can define an internal lumen. The internal lumen of threaded portion 126 can be in fluid communication with the internal lumen of gripping portion 122.

Stop 128 can be located between threaded portion 126 and gripping portion 122. In some cases, stop 128 can have a diameter greater than threaded portion 126 and/or gripping portion 122. In some case, stop 128 can limit rotation of cap 140 during attachment of cap 140 to elongated shaft 120 (e.g., via threaded portion 126). In some cases, stop 128 can limit movement of a hand of user 100 past stop 128 when using tissue collection device 110. For example, when user 100 applies pressure to tissue collection device 110 to scrape tissue sample 106, the fingers of user 100 can slide down the gripping portion 122, toward the surface on which the tissue sample 106 is located. The fingers of user 100 can abut stop 128 to limit further distal movement of the fingers along tissue collection device 110. Stop 128 can aid in protecting the user from interacting with cap 140, which can include a blade 148.

Filter column 130 can be received by elongated shaft 120. Filter column 130 can include a main column 132, a filter end 134, a coupling area 136, and a lip 138. Filter column 130 can be made of any suitable material such as, but not limited to, polypropylene, xylene-resistant material, ethanol-resistant material, DNase-free material, and/or RNase-free material.

Filter end 134 can include a filter member/element. In some cases, the filter member/element can be coupled to the filter end 134. In some cases, the filter member/element can be removably coupled to filter end 134. Filter end 134 can be sized and shaped to allow filter column 130 to be received in an internal lumen of elongated shaft 120. In some cases, the filter member/element can be a mesh filter. In some cases, the filter member/element can be made of polypropylene. In some cases, the filter member/element can have a pore size of 15-35 μm (e.g., 25 μm, 30 μm). In some cases, the mesh filter can have a diameter of 15-35 mm (e.g., 25 mm).

Main column 132 can include a central lumen. The central lumen of main column 132 can receive/trap particles that do not pass through the filter member/element at filter end 134. Main column 132 can be sized and shaped to be received by the internal lumen of elongated shaft 120. Further, main column 132 can be removed from the internal lumen of elongated shaft 120 (e.g., once scraping has been completed and a vacuum has been turned off) with the flakes collected from scraping tissue sample 106 from slide 102. In some cases, main column 132 can be sized and shaped to allow main column 132, and correspondingly filter column 130, to be received by a centrifuge tube, such that the flakes collected from tissue sample 106 can be processed for nucleic acid extraction. Main column 132 can be used to gravity feed various chemicals (e.g., xylene, ethanol, etc.) through filter column 130. These chemicals can be washed past the tissue sample 106 that was collected on the filter of filter column 130.

Coupling area 136 can be sized and shaped to allow filter column 130 to couple with cap 140. In some cases, coupling area 136 can have a diameter wider than main column 132. In this case, coupling area 136 can receive a portion of cap 140 inside coupling area 136. In some cases, the difference in diameter between coupling area 136 and main column 132 can correspond to a diameter of the portion of cap 140 that the coupling area 136 receives, such that an internal diameter of the portion of cap 140 is substantially similar to an internal diameter of main column 132. In some cases, coupling area 136 can couple to cap 140 via a snug fit due to a relationship between the diameters of the coupling area 136 and the portion of cap 140 that couples to filter column 130. In some cases, coupling area 136 can be threaded to provide screw-like coupling between filter column 130 and cap 140.

Lip 138 can be sized to prevent filter column 130 from being completely received in elongated shaft 120. For example, lip 138 can have a diameter greater than a diameter of the internal lumen of elongated shaft 120. In some cases, lip 138 can also receive a cover. The cover can provide an enclosed area to limit loss of particles when transporting filter column 130. The cover can also provide a sealed container to allow various reactions described herein to take place. In some cases, lip 138 can aid in securing filter column 130 and cap 140 together.

In some cases, tissue collection device 110 can be disposable (e.g., after a single use). In some cases, tissue collection device 110 can be packaged in a kit. In some cases, tissue collection device 110 can be assembled before use. In some cases, assembly can include placing an empty filter column 130 into the lumen defined by threaded portion 126, with the filter end being received by elongated shaft 120 first. In some cases, assembly can include placing a filter in filter column 130 (e.g., before or after inserting filter column 130 into elongated shaft 120). In some cases, assembly can include screwing cap 140 onto threaded portion 126 of elongated shaft 120 until cap 140 reaches stop 128. In some cases, vacuum tubing can be attached to vacuum coupler 124 during assembly. In some cases, vacuum pressure can be turned on to collect tissue fragments from tissue sample 106 when scraping tissue sample 106 with tissue collection device 110. In some cases, the filter column, with tissue fragments, can be removed from elongated shaft 120 and placed into a microcentrifuge tube. In some cases, the microcentrifuge tube can be used for deparaffinization, proteinase K digestion, and/or DNA extraction.

Referring to FIGS. 4 and 5, cap 140 can include a threaded portion 142, a central lumen 144, an extension portion 146, and a blade 148. Cap 140 can be made of polypropylene, xylene-resistant material, ethanol-resistant material, DNase-free material, and/or RNase-free material.

Threaded portion 142 can include internal threads to allow coupling between cap 140 and threaded portion 126 of elongated shaft 120. An exterior diameter of threaded portion 142 may be substantially similar to elongated shaft 120. In some cases, the exterior diameter of threaded portion 142 is substantially similar to stop 128, while in other cases, the exterior diameter of threaded portion 142 is substantially similar to gripping portion 122. Threaded portion 142 of cap 140 can secure filter column 130 inside elongated shaft 120. In some cases, threaded portion 142 can create a vacuum seal between cap 140 and elongated shaft 120, allowing suction to pass through the entirety of tissue collection device 110. In some cases, threaded portion 142 can include a seal to increase the vacuum seal created between cap 140 and elongated shaft 120.

Central lumen 144 can be defined by a cylindrical component of cap 140 located in cap 140 longitudinally. Central lumen 144 can extend substantially an entire length of cap 140, such that a gap is created between the cylindrical component defining central lumen 144 and threaded portion 142. In some cases, central lumen 144 may not extend all the way to a free edge of the threaded portion 142 that abuts elongated shaft 120. Central lumen 144 can provide passage of flakes of tissue sample 106 from the slide 102 to the filter column 130. In some cases, central lumen 144 can have a substantially constant diameter along the length of central lumen 144. In some cases, the diameter of central lumen 144 can taper as central lumen 144 approaches blade 148. In some cases, central lumen 144 has an internal diameter substantially similar to an internal diameter of filter column 130 (e.g., main column). In some cases, the internal diameter of central lumen 144 can be smaller than the internal diameter of filter column 130 (e.g., main column 132). In some cases, coupling area 136 of filter column 130 can be securely coupled to (e.g., around) the cylindrical component that defines the central lumen 144.

Extension portion 146 can extend between threaded portion 142 and blade 148. Extension portion 146 can provide an area for a user to grip cap 140 when securing cap 140 to elongated shaft 120. In some cases, extension portion 146 can be conical shaped. The conical shape can reduce the obstruction of vision of the blade 148 when using tissue collection device 110. In some cases, a proximal external diameter of extension portion 146 can be substantially similar to an external diameter of threaded portion 142. In some cases, a proximal external diameter of extension portion 146 can be smaller than an external diameter of threaded portion 142. In some cases, extension portion 146 can be cylindrically shaped.

Blade 148 can be used to scrape tissue sample 106 from slide 102. As flakes are created from scraping tissue sample 106 from slide 102, vacuum suction through tissue collection device 110 can remove the flakes from slide 102 and collect the flakes in the filter column 130. In some cases, blade 148 can be made of a material similar to the rest of cap 140. Alternatively, blade 148 can be made of a different material than the rest of cap 140. In some cases, blade 148 can be located along a central axis of central lumen 144. Alternatively, blade 148 can be offset from central lumen 144, such as along an edge of central lumen 144. In some cases, blade 148 can include a single sharp edge. In some cases, blade 148 can include multiple sharp edges. In some cases, edges of blade 148 can come to a point. In some cases, sides of blade 148 can create a flat edge between the sides to create a wider tip. In some cases, one side of the blade can have an angle such that when the tissue collection device 110 is held similar to a writing utensil, or other small handheld devices, the side is flush with the surface (e.g., slide 102). In some cases, the angle can create an edge for scraping tissue sample 106 from slide 102. In some cases, a side of blade 148 can be angled to create an acute angle with a surface (e.g., slide 102) when the tissue collection device 110 is held similar to a writing utensil, or other small handheld devices, the side is flush with the surface (e.g., slide 102). In some cases, one or more edges of blade 148 can be flush with a diameter of extension portion 146. In some cases, the width of blade 148 can be substantially similar for the entire blade 148. In some cases, the width of blade 148 may vary (e.g., increases towards extension portion 146). In some cases, blade 148 can be triangular, square, rectangular, or other shape sufficient to couple with extension portion 146 and allow for scraping.

Referring to FIGS. 6 and 7, a cap 150 can include a threaded portion 152, a central lumen 154, an extension portion 156, and a blade 158. Cap 150 can be made of polypropylene, xylene-resistant material, ethanol-resistant material, DNase-free material, and/or RNase-free material.

Threaded portion 152 can include internal threads to allow coupling between cap 150 and threaded portion 126 of elongated shaft 120. An exterior diameter of threaded portion 152 may be substantially similar to elongated shaft 120. In some cases, the exterior diameter of threaded portion 152 is substantially similar to stop 128, while in other cases, the exterior diameter of threaded portion 152 is substantially similar to gripping portion 122. Threaded portion 152 of cap 140 can secure filter column 130 inside elongated shaft 120. In some cases, threaded portion 152 can create a vacuum seal between cap 150 and elongated shaft 120, allowing suction to pass through the entirety of tissue collection device 110. In some cases, threaded portion 152 can include a seal to increase the vacuum seal created between cap 150 and elongated shaft 120.

Central lumen 154 can be defined by a cylindrical component of cap 150 located in cap 150 longitudinally. Central lumen 154 can extend substantially an entire length of cap 150, such that a gap is created between the cylindrical component defining central lumen 154 and threaded portion 152. In some cases, central lumen 154 may not extend all the way to a free edge of the threaded portion 152 that abuts elongated shaft 120. Central lumen 154 can provide passage of flakes of tissue sample 106 from the slide 102 to the filter column 130. In some cases, central lumen 154 can have a substantially constant diameter along the length of central lumen 154. In some cases, the diameter of central lumen 154 can taper as central lumen 154 approaches blade 158. In some cases, central lumen 154 has an internal diameter substantially similar to an internal diameter of filter column 130 (e.g., main column 132). In some cases, the internal diameter of central lumen 154 can be smaller than the internal diameter of filter column 130 (e.g., main column 132). In some cases, coupling area 136 of filter column 130 can be securely coupled to (e.g., around) the cylindrical component that defines the central lumen 154.

Extension portion 156 can extend between threaded portion 152 and blade 158. Extension portion 156 can provide an area for a user to grip cap 150 when securing cap 150 to elongated shaft 120. In some cases, extension portion 156 can be conical shaped. The conical shape can reduce the obstruction of vision of the blade 158 when using tissue collection device 110. In some cases, a proximal external diameter of extension portion 156 can be substantially similar to an external diameter of threaded portion 152. In some cases, a proximal external diameter of extension portion 156 can be smaller than an external diameter of threaded portion 152. In some cases, extension portion 156 can be cylindrically shaped.

Blade 158 can be used to scrape tissue sample 106 from slide 102. As flakes are created from scraping tissue sample 106 from slide 102, vacuum suction through tissue collection device 110 can remove the flakes from slide 102 and collect the flakes in the filter column 130. In some cases, blade 158 can be made of a material similar to the rest of cap 150. Alternatively, blade 158 can be made of a different material than the rest of cap 150. In some cases, blade 158 can be located along a central axis of central lumen 154. Alternatively, blade 158 can be offset from central lumen 154, such as along an edge of central lumen 154. In some cases, blade 158 can include a single sharp edge. In some cases, blade 158 can include multiple sharp edges. In some cases, the edges of blade 158 can come to a point. In some cases, the sides of blade 158 can create a flat edge between the sides to create a wider tip. In some cases, one side of the blade can have an angle such that when the tissue collection device 110 is held similar to a writing utensil, or other small handheld devices, the side is flush with the surface (e.g., slide 102). In some cases, the angle can create an edge for scraping tissue sample 106 from slide 102. In some cases, a side of blade 158 can be angled to create an acute angle with a surface (e.g., slide 102) when the tissue collection device 110 is held similar to a writing utensil, or other small handheld devices, the side is flush with the surface (e.g., slide 102). In some cases, one or more edges of blade 158 can be flush with a diameter of extension portion 156. In some cases, the width of blade 158 can be substantially similar for the entire blade 158. In some cases, the width of blade 158 may vary (e.g., increases towards extension portion 156). In some cases, blade 158 can be triangular, square, rectangular, or other shape sufficient to couple with extension portion 156 and allow for scraping.

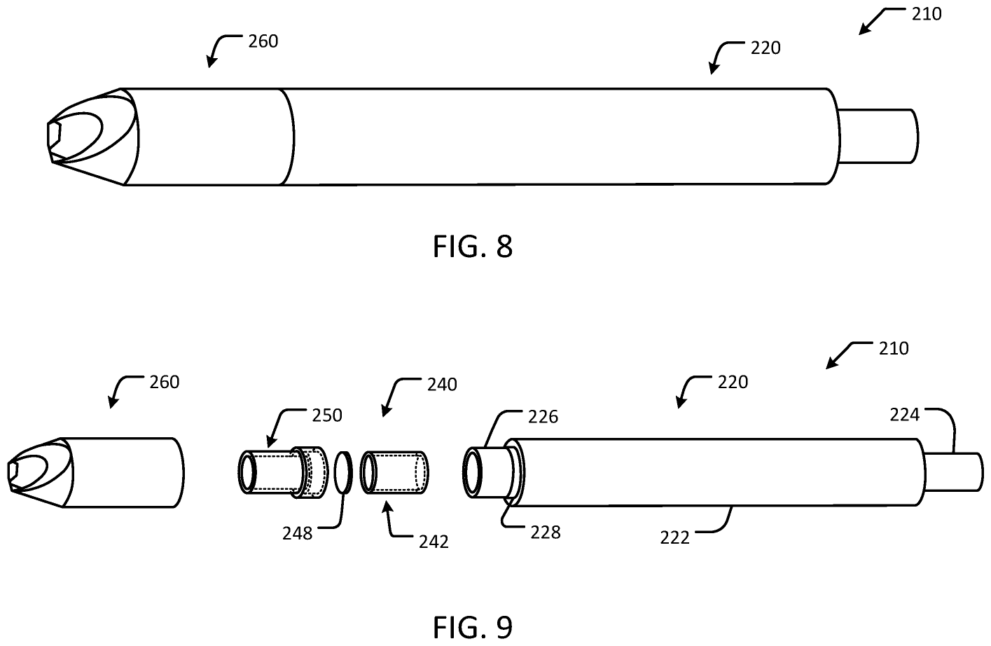

Referring to FIGS. 8-16, tissue collection device 210 can include an elongated shaft 220, a filter assembly 240, and a cap 260. In some cases, tissue collection device 210 can be made via injection molding, machining, and/or 3D printing. In some cases, parts or all of tissue collection device 210 can be reusable.

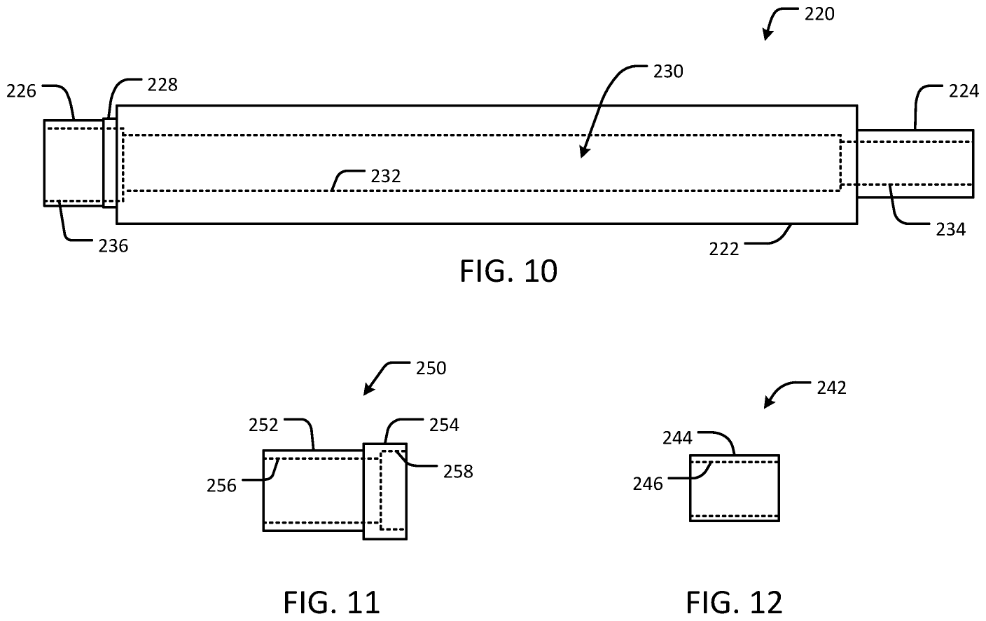

Referring to FIG. 10, elongated shaft 220 can include a gripping portion 222, a vacuum coupler 224, a cap coupler 226, and a ridge 228. Elongated shaft 220 can also include an internal lumen 230. Elongated shaft 220 can be made of polypropylene, acetal (e.g., Delrin), polyether ether ketone (PEEK), stainless steel, xylene-resistant material, ethanol-resistant material, DNase-free material, and/or RNase-free material.

Gripping portion 222 can be cylindrical. In some cases, gripping portion 222 can be a rectangular prism, a triangular prism, or other prism form. In some cases, gripping portion 222 can be sized to comfortably fit in a hand of user 100. Gripping portion 222 can define a gripping portion internal lumen 232. In some cases, gripping portion 222 can include at least one of ridges, texture, indentations, and/or a portion of different material. In some cases, these features can increase coupling and/or comfort between the user 100 and the tissue collection device 210.

Vacuum coupler 224 can be located at a proximal end of gripping portion 222. Vacuum coupler 224 can be configured to receive a tube or other component of a vacuum, or other suction device. Vacuum coupler 224 can allow vacuum suction to be provided to the tissue collection device 210 to aid in collection of tissue. In some cases, the tube of a vacuum can be fitted around vacuum coupler 224. In some cases, vacuum coupler 224 can define a vacuum coupler internal lumen 234. The vacuum coupler internal lumen 234 is in fluid communication with gripping portion internal lumen 232. In some cases, vacuum coupler 224 can threaded for a screw-type connection with a vacuum or suction device.

Cap coupler 226 can be located at a distal end of gripping portion 222. In some cases, cap coupler 226 can have a diameter smaller than that of gripping portion 222. In some cases, cap coupler 226 can define a cap coupler internal lumen 236. In some cases, the cap coupler internal lumen 236 can have a larger diameter than gripping portion internal lumen 232 such that a counterbore is created. The cap coupler internal lumen 236 can be in fluid communication with gripping portion internal lumen 232.

Ridge 228 can be created between cap coupler 226 and gripping portion 222. In some cases, ridge 228 can have a diameter greater than cap coupler 226 smaller than gripping portion 222. In some case, ridge 228 can aid in securing cap 260 during attachment of cap 260 to elongated shaft 220.

Referring to FIGS. 11 and 12, filter assembly 240 can be received by elongated shaft 220 and/or cap 260. Filter assembly 240 can include a male filter column 242, a filter 248, and a female filter column 250. Filter assembly 240, and components of the tissue collection device 210 can be sized and shaped such that the filter assembly 240 can fit in tissue collection device 210 in a single orientation. Male filter column 242 can be a cylindrical column 244 defining a male filter internal lumen 246. The male filter column 242 can have a diameter such that male filter column 242 can be received in the counterbore defined by cap coupler internal lumen 236. The internal diameter of cap coupler internal lumen 236 and the external diameter of male filter cylindrical column 244 can be sized such that the male filter 242 can be secured in the cap coupler internal lumen 236 with a press fit.

Filter 248 can be removably coupled between male filter column 242 and female filter column 250. In some cases, filter 248 can be for a single use. In some cases, filter 248 can be a mesh filter. In some cases, filter 248 can be made of polypropylene. In some cases, filter 248 can be chemical-resistant. In some cases, filter 248 can have a pore size of 20-80 μm (e.g., 25 μm, 30 μm, 40 μm, 50 μm, etc.). In some cases, filter 248 can have a diameter of 6-12 mm (e.g., 10 mm).

In some cases, female filter column 250 can include a main body 252 and a coupling portion 254. The female filter column main body 252 can define a female filter internal lumen 256. In some cases, the coupling portion 254 can define a coupling portion internal lumen 258. In some cases, the coupling portion internal lumen 258 and the female filter internal lumen 256 can be sized such that a counterbore is created between the coupling portion internal lumen 258 and the female filter internal lumen 256. The coupling portion internal lumen 258 can receive filter 248 and male filter 242. In some cases, the coupling portion internal lumen 258 can secure the filter 248 and male filter 242 with a press fit due to the interface between the coupling portion internal lumen 258 and the male filter 242.

Filter assembly 240 can be made of any suitable material such as, but not limited to, polypropylene, acetal (e.g., Delrin), polyether ether ketone (PEEK), xylene-resistant material, ethanol-resistant material, DNase-free material, and/or RNase-free material. In some cases, once the filter assembly 240 has collected tissue samples, filter assembly 240 can be used for deparaffinization, proteinase K digestion, and/or DNA extraction. In some cases, filter assembly 240 can be placed in a tube and various chemicals can be gravity fed through filter 248.

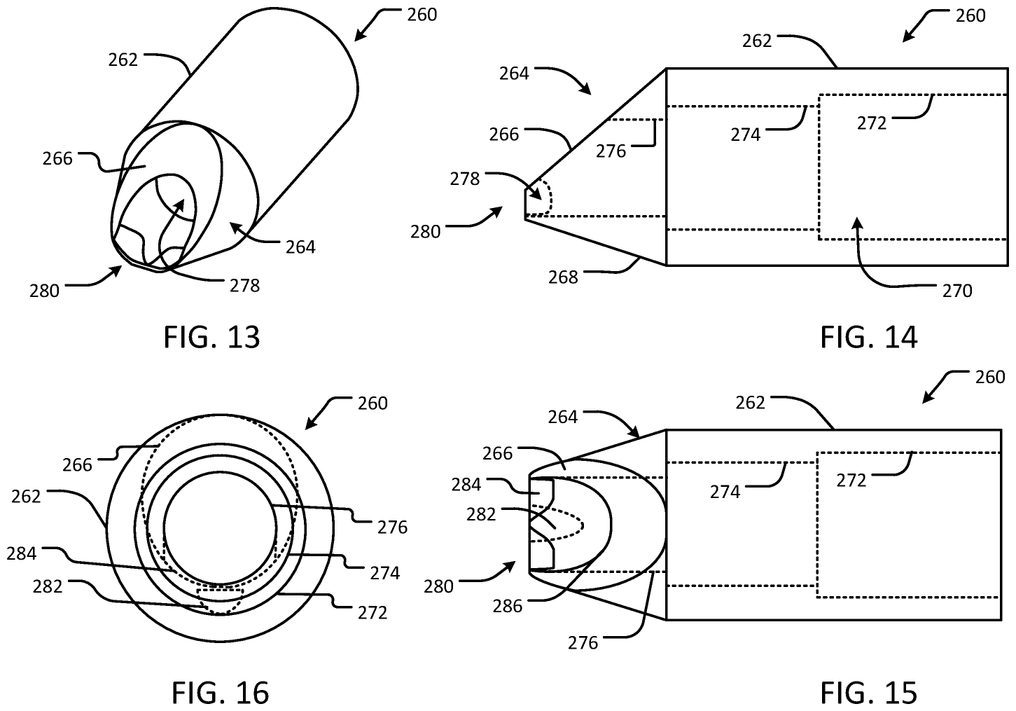

Referring to FIGS. 13-16, cap 260 can include a cylindrical portion 262, a conical portion 264, a cap internal lumen 270, and a blade portion 280. Cap 260 can be made of polypropylene, acetal (e.g., Delrin), polyether ether ketone (PEEK), polycarbonate, acrylonitrile butadiene styrene (ABS), xylene-resistant material, ethanol-resistant material, DNase-free material, and/or RNase-free material.

Cylindrical portion 262 can define a first internal lumen 272 and a second internal lumen 274. First internal lumen 272 can have a first diameter and second internal lumen 274 can have a second diameter greater than the first diameter, such that a counterbore is created. The first internal lumen 272 and/or the second internal lumen 274 can be sized to receive the main body 252 of the female filter column 250. In some cases, the first internal lumen 272 can be sized to receive the cap coupler 226 of elongated shaft 220. An exterior diameter of cylindrical portion 262 may be substantially similar to elongated shaft 220, such as gripping portion 222.

Conical portion 264 can include an upper slope 266, a lower slope 268, and can define a conical internal lumen 276. In some cases, the upper slope 266 can have an angle of 30-50 degrees from an upper horizontal. In some cases, the upper slope 266 is a flat surface. In some cases, the lower slope 268 can have an angle of 10-20 degrees from a lower horizontal. In some cases, the diameter of conical internal lumen 276 can taper as conical internal lumen 276 approaches blade portion 280. In some cases, conical internal lumen 276 can have an internal diameter smaller than the internal diameter of second internal lumen 274.

Blade portion 280 can be used to scrape tissue sample 106 from slide 102. As flakes are created from scraping tissue sample 106 from slide 102, vacuum suction through tissue collection device 210 can remove the flakes from slide 102 and collect the flakes in the filter assembly 240. In some cases, blade portion 280 can be made of a material similar to the rest of cap 260. In some cases, blade portion 280 can include a large opening 278. In some cases, a large opening can allow larger scraping to be collected in tissue collection device 210. In some cases, blade portion 280 can include a first blade 282, a second blade 284, and/or a third blade 286. In some cases, first blade 282 can have a curvature that can provide a select area of scraping. In some cases, the first blade 282 can provide various areas of scraping based on an angle of tissue collection device 210. In some cases, second blade 284 can have a wider surface than first blade 282. In some cases, second blade 284 can be a similar to a flat shovel. In some cases, second blade 284 can come to a point to create a blade for smaller areas. In some cases, some, or all, of blade portion 280 can have a width of one tenth of an inch. In some cases, third blade 286 can surround a circumference of upper slope 266.

In some cases, some, or all, of tissue collection device 210 can be disposable (e.g., after a single use). In some cases, some, or all, of tissue collection can be used multiple times. In some cases, tissue collection device 210 can be packaged in a kit. In some cases, tissue collection device 210 can be assembled before use. In some cases, assembly can include placing an empty filter assembly 240 into the lumens defined by cap coupler 226 and cap 260. In some cases, assembly can include securing cap 260 onto cap coupler 226 of elongated shaft 220 until cap 260 reaches ridge 228. In some cases, vacuum tubing can be attached to vacuum coupler 224 during assembly. In some cases, vacuum pressure can be turned on to collect tissue fragments from tissue sample 106 when scraping tissue sample 106 with tissue collection device 210. In some cases, the filter assembly 240, or portions thereof, with tissue fragments, can be removed from elongated shaft 220 and/or cap 260, and placed into a microcentrifuge tube. In some cases, the microcentrifuge tube can be used for deparaffinization, proteinase K digestion, and/or DNA extraction.

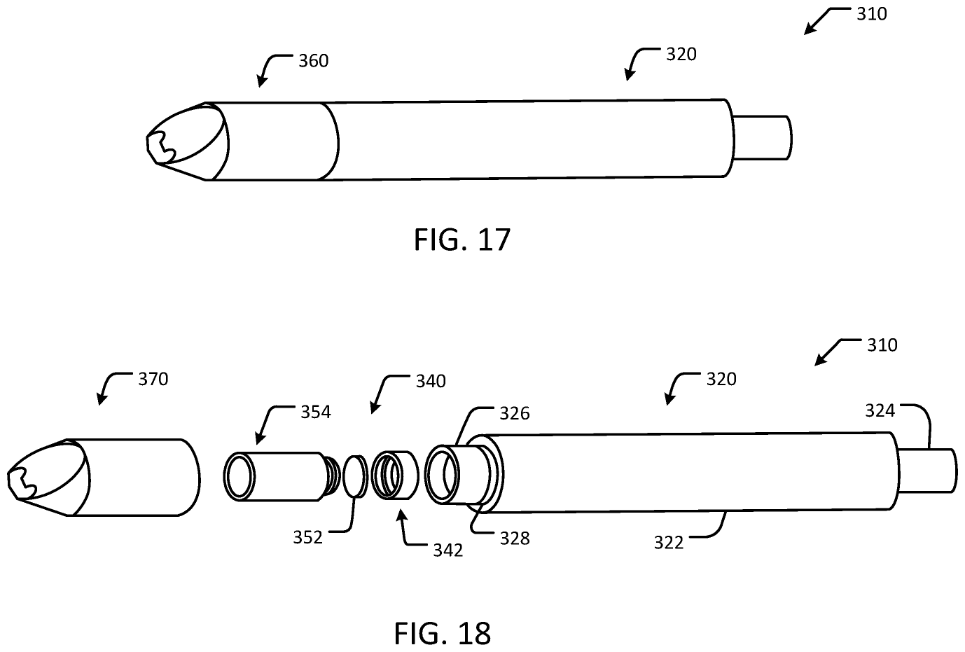

Referring to FIGS. 17-25, tissue collection device 310 can include an elongated shaft 320, a filter assembly 340, and a cap 370. In some cases, tissue collection device 310 can be made via injection molding, machining, and/or 3D printing. In some cases, parts or all of tissue collection device 310 can be reusable.

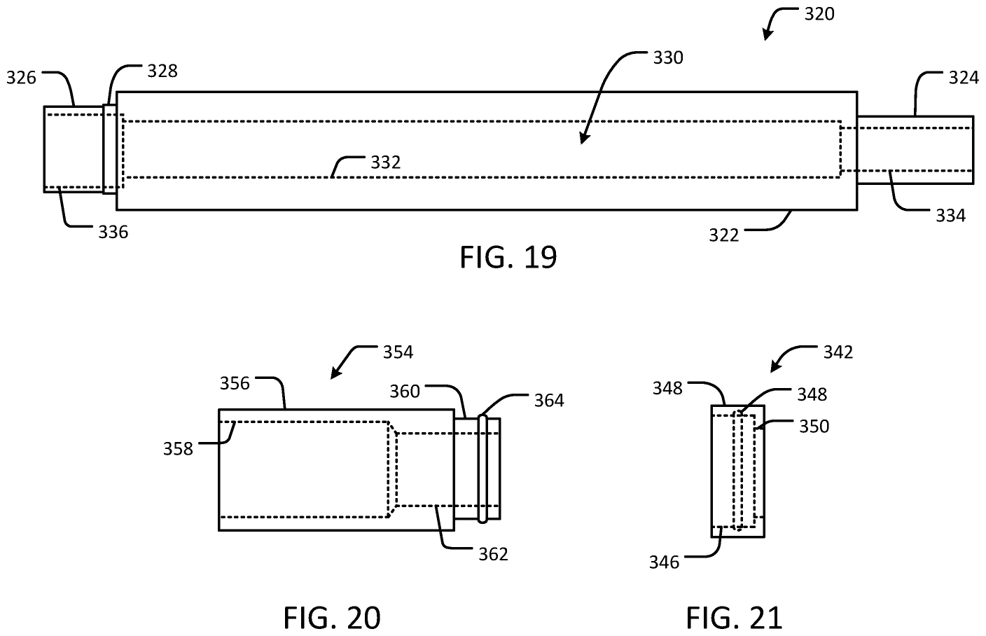

Referring to FIG. 19, elongated shaft 320 can be substantially similar to elongated shaft 220. Elongated shaft 320 can include a gripping portion 322, a vacuum coupler 324, a cap coupler 326, and a ridge 328. Elongated shaft 320 can also include an internal lumen 330. Elongated shaft 320 can be made of polypropylene, acetal (e.g., Delrin), polyether ether ketone (PEEK), stainless steel, xylene-resistant material, ethanol-resistant material, DNase-free material, and/or RNase-free material.

Gripping portion 322 can be cylindrical. In some cases, gripping portion 322 can be a rectangular prism, a triangular prism, or other prism form. In some cases, gripping portion 322 can be sized to comfortably fit in a hand of user 100. Gripping portion 322 can define a gripping portion internal lumen 332. In some cases, gripping portion 322 can include at least one of ridges, texture, indentations, and/or a portion of different material. In some cases, these features can increase coupling and/or comfort between the user 100 and the tissue collection device 310.

Vacuum coupler 324 can be located at a proximal end of gripping portion 322. Vacuum coupler 324 can be configured to receive a tube or other component of a vacuum, or other suction device. Vacuum coupler 324 can allow vacuum suction to be provided to the tissue collection device 310 to aid in collection of tissue. In some cases, the tube of a vacuum can be fitted around vacuum coupler 324. In some cases, vacuum coupler 324 can define a vacuum coupler internal lumen 334. The vacuum coupler internal lumen 334 is in fluid communication with gripping portion internal lumen 332. In some cases, vacuum coupler 324 can threaded for a screw-type connection with a vacuum or suction device.

Cap coupler 326 can be located at a distal end of gripping portion 322. In some cases, cap coupler 326 can have a diameter smaller than that of gripping portion 322. In some cases, cap coupler 326 can define a cap coupler internal lumen 336. In some cases, the cap coupler internal lumen 336 can have a larger diameter than gripping portion internal lumen 332 such that a counterbore is created. The cap coupler internal lumen 336 can be in fluid communication with gripping portion internal lumen 332. In some cases, cap coupler 326 can include threads to couple with cap 370.

In some cases, ridge 328 can be created between cap coupler 326 and gripping portion 322. In some cases, ridge 328 can have a diameter greater than cap coupler 326 and smaller than gripping portion 322. In some case, ridge 328 can aid in securing cap 370 during attachment of cap 370 to elongated shaft 320. For example, ridge 328 can create a snap-fit between cap 370 and cap coupler 326. As another example, ridge 328 can create an interference fit between cap 370 and cap coupler 326.

Referring to FIGS. 20 and 21, filter assembly 340 can be received by elongated shaft 320 and/or cap 370. For example, filter assembly 340 can have a length such that filter assembly 340 extends from cap 370 to elongated shaft 320. Filter assembly 240 can include a female filter column 342, a filter 352, and a male filter column 354. In some cases, a diameter of filter assembly 340 can be smaller than filter assembly 240. Filter assembly 340 can be sized such that filter assembly 340 can fit into a microcentrifuge tube, and when solution is placed in the microcentrifuge tube, filter 352 is submerged in the solution, increasing the amount of DNA that can be removed from the filter, and therefore the recovery rate of the scraped tissue.

In some cases, male filter column 354 can include a cylindrical main body 356 and a coupling portion 360. The main body 356 can define a male filter internal lumen 358. The male filter column 354 can have a diameter such that male filter column 354 can be received by cap 370. An internal diameter of cap 370 and the external diameter of male filter cylindrical column 356 can be sized such that the male filter 354 can be secured in the cap 370 with a press fit. In some cases, the coupling portion 360 can define a coupling portion internal lumen 362. In some cases, the coupling portion 360 includes a ridge 364. As another example, the coupling portion 360 can include one or more threads configured to engage with female filter column 342.

Filter 352 can be removably coupled between female filter column 342 and male filter column 354. In some cases, filter 352 can be for a single use. In some cases, filter 352 can be a mesh filter. In some cases, filter 352 can be made of polypropylene. In some cases, filter 352 can be chemical-resistant. In some cases, filter 352 can have a pore size of 20-80 μm (e.g., 25 μm, 30 μm, 40 μm, 50 μm, etc.). In some cases, filter 352 can have a diameter of 6-12 mm (e.g., 10 mm). In some cases, filter 352 can have a diameter substantially similar to coupling portion 360.

Female filter column 342 can be a cylindrical column 344 defining a female filter internal lumen 346. Cylindrical column 344 can have a diameter such that female filter column 342 can be received by cap coupler internal lumen 336. In some cases, the diameter of cylindrical column 344 can be slightly larger than the internal diameter of cap coupler internal lumen 336, creating an interference fit. The female filter internal lumen 346 can have a diameter such that the coupling portion 360 of male filter column 354 can be received by female filter internal lumen 346. In some cases, the diameter of female filter internal lumen 346 can be slightly smaller than the diameter of coupling portion 360 of male filter column 354, creating an interference fit. In some cases, the diameter of female filter internal lumen 346 can be substantially similar to the diameter of filter 352. In some cases, female filter internal lumen 346 can include a recess 348 that is configured to receive ridge 364. Recess 348 can aid in securing male filter column 354 and female filter column 342 together. As another example, female filter internal lumen 346 can include one or more threads configured to engage with male filter column 354. In some cases, female filter column 342 can include a second internal lumen 350. Second internal lumen 350 can have a diameter smaller than female filter internal lumen 346, creating a ring shaped ridge between female filter internal lumen 346 and second internal lumen 350. Accordingly, restricting forces are applied from an end of coupling portion 360 of male filter column 354, and from the ring shaped ridge of the female filter column 342. These forces act towards each other against the outer edges of the outer surfaces of the filter 352 when a proximal end of cylindrical main body 356 and a distal end of cylindrical column 344 contact, thereby immobilizing filter 352 and preventing tissue from breaching the interface between male filter column 354 and female filter column 342.

Filter assembly 340 can be made of any suitable material such as, but not limited to, polypropylene, acetal (e.g., Delrin), polyether ether ketone (PEEK), xylene-resistant material, ethanol-resistant material, DNase-free material, and/or RNase-free material. In some cases, once the filter assembly 340 has collected tissue samples, filter assembly 340 can be used for deparaffinization, proteinase K digestion, and/or DNA extraction. In some cases, filter assembly 340 can be placed in a tube and various chemicals can be gravity fed through filter 348. For example, filter assembly 340 can be placed in a microcentrifuge tube.

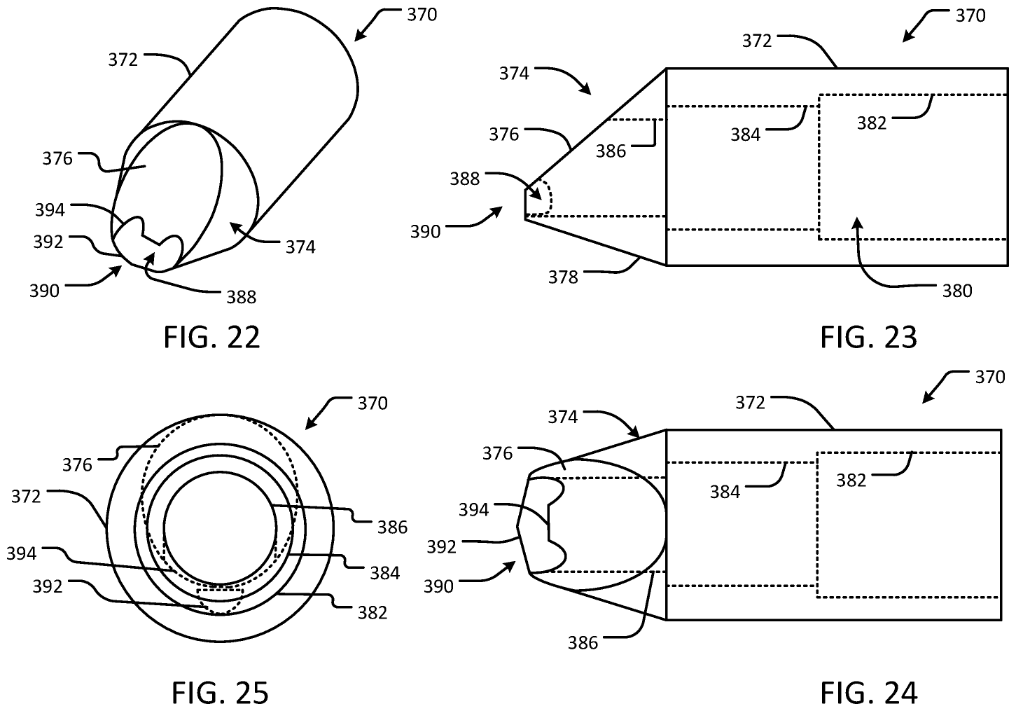

Referring to FIGS. 22-25, cap 370 can include a cylindrical portion 372, a conical portion 374, a cap internal lumen 380, and a blade portion 390. Cap 370 can be made of polypropylene, acetal (e.g., Delrin), polyether ether ketone (PEEK), polycarbonate, acrylonitrile butadiene styrene (ABS), xylene-resistant material, ethanol-resistant material, DNase-free material, and/or RNase-free material.

Cylindrical portion 372 can define a first internal lumen 382 and a second internal lumen 384. First internal lumen 382 can have a first diameter and second internal lumen 384 can have a second diameter greater than the first diameter, such that a counterbore is created. The first internal lumen 382 and/or the second internal lumen 384 can be sized to receive the main body 356 of the male filter column 354. In some cases, the first internal lumen 382 can be sized to receive the cap coupler 326 of elongated shaft 320. In some cases, the first internal lumen 382 can include threading to couple with the cap coupler 326. An exterior diameter of cylindrical portion 342 may be substantially similar to elongated shaft 320, such as gripping portion 322.

Conical portion 374 can include an upper slope 376, a lower slope 378, and can define a conical internal lumen 386. In some cases, the upper slope 376 can have an angle of 30-50 degrees from an upper horizontal. In some cases, the upper slope 376 is a flat surface. In some cases, the lower slope 378 can have an angle of 10-20 degrees from a lower horizontal. In some cases, the diameter of conical internal lumen 386 can taper as conical internal lumen 386 approaches blade portion 390. In some cases, conical internal lumen 386 can have an internal diameter smaller than the internal diameter of second internal lumen 384.

Blade portion 390 can be used to scrape tissue sample 106 from slide 102. As flakes are created from scraping tissue sample 106 from slide 102, vacuum suction through tissue collection device 310 can remove the flakes from slide 102 and collect the flakes in the filter assembly 340. In some cases, blade portion 390 can be made of a material similar to the rest of cap 370. In some cases, blade portion 390 can include an opening 388. In some cases, opening 388 can be sized and/or shaped to allow larger scraping to be collected in tissue collection device 310. In some cases, blade portion 390 can include a first blade 392 and/or a second blade 394. In some cases, first blade 392 can have a curvature that can provide a select area of scraping. For example, first blade 392 can have a point. In some cases, the first blade 392 can provide various areas of scraping based on an angle of tissue collection device 310. In some cases, second blade 394 can have a wider surface than first blade 392. In some cases, second blade 394 can be a similar to a flat shovel. In some cases, second blade 394 can surround the opening 388 formed by upper slope 376. In some cases, second blade 394 is just a portion of upper slope 376 and does not act as a blade. In some cases, some, or all, of blade portion 390 can have a width of one tenth of an inch.

In some cases, some, or all, of tissue collection device 310 can be disposable (e.g., after a single use). In some cases, some, or all, of tissue collection can be used multiple times. In some cases, tissue collection device 310 can be packaged in a kit. In some cases, tissue collection device 310 can be assembled before use. In some cases, assembly can include placing an empty filter assembly 340 into the lumens defined by cap coupler 326 and cap 370. In some cases, assembly can include securing cap 370 onto cap coupler 326 of elongated shaft 320 until cap 370 reaches ridge 328. In some cases, vacuum tubing can be attached to vacuum coupler 324 during assembly. In some cases, vacuum pressure can be turned on to collect tissue fragments from tissue sample 106 when scraping tissue sample 106 with tissue collection device 310. In some cases, the filter assembly 340, or portions thereof, with tissue fragments, can be removed from elongated shaft 320 and/or cap 370, and placed into a tube, such as a microcentrifuge tube. In some cases, the microcentrifuge tube can be used for deparaffinization, proteinase K digestion, and/or DNA extraction.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the process depicted in the accompanying figures do not necessarily require the particular order shown, or sequential order, to achieve desirable results. In certain implementations, multitasking and parallel processing may be advantageous.

What is claimed is:

1. A sample collection device, the device comprising:
an elongated shaft defining an internal lumen and comprising a proximal end and a distal end, wherein the proximal end of the elongated shaft is configured to couple with a suction source;
a cap defining an internal lumen and comprising a proximal end and a distal end, wherein the internal lumen of the cap is fluidly coupled to the internal lumen of the elongated shaft, wherein the proximal end of the cap is configured to couple to the distal end of the elongated shaft, and wherein the distal end of the cap comprises a blade; and
a filter column configured to be secured inside the internal lumen of the elongated shaft via the cap.

2. The device of claim 1, wherein the second end of the elongated shaft and the first end of the cap are threaded.

3. The device of claim 1, wherein the filter column includes a filter member.

4. The device of claim 3, wherein the filter member is located on a distal end of the filter column.

5. The device of claim 1, wherein the filter column is configured to collect a tissue sample.

6. The device of claim 1, wherein the proximal end of the cap is further configured to removably couple to the filter column.

7. The device of claim 5, wherein a proximal end of the filter column is configured to removably couple to the proximal end of the cap.

8. The device of claim 1, wherein the sample is a tissue sample.

9. A method of collecting a sample, the method comprising:
inserting a filter column into a distal end of internal lumen of an elongated shaft; and
securing a cap comprising a blade onto a distal end of the elongated shaft.

10. The method of claim 9, further comprising coupling a suction source to a proximal end of the elongated shaft.

11. The method of claim 9, further comprising using the blade to remove portions of the sample.

12. The method of claim 11, further comprising applying suction to the internal lumen to suck the portions of the sample into the filter column.

13. The method of claim 12, wherein the portions of the sample are caught in a filter member located in the filter column.

14. The method of claim 9, further comprising removing the filter column from the internal lumen with portions of the sample inside the filter column.

15. A method of collecting a sample, the method comprising:
scraping the sample using a blade to create portions of the sample; and
sucking the portions into a filter column of a tissue collection device,
wherein the tissue collection device comprises:
an elongated shaft defining an internal lumen and comprising a proximal end and a distal end, wherein the proximal end of the elongated shaft is configured to couple to a suction source;
a cap defining an internal lumen and comprising a proximal end and a distal end, wherein the proximal end of the cap is configured to couple to the distal end of the elongated shaft, and wherein the distal end of the cap comprises a blade; and
a filter column configured to be secured inside the internal lumen of the elongated shaft via the cap.

16. The method of claim 15, further comprising trapping the portions of the sample in the filter column.

17. The method of claim 16, further comprising removing the cap from the elongated shaft.

18. The method of claim 17, further comprising removing the filter column from the internal lumen of the elongated shaft.

19. The method of claim 18, further comprising inserting the filter column into a centrifuge tube.

20. The method of claim 17, further comprising performing molecular testing in the filter column.
